(19) United States
(12) Plant Patent Application Publication
(10) Pub. No.: US 2012/0204302 P1
(43) Pub. Date: Aug. 9, 2012

(54) Gerbera plant named 'UFGE 7014'

(50) ;

(76) Inventors: Zhanao Deng, Riverview, FL (US); Brent K. Harbaugh, (US)

(21) Appl. No.: 12/931,609

(22) Filed: Feb. 4, 2011

(51) Int. Cl. A01H 5/00

(52) U.S. Cl. PLT/357

(57) ABSTRACT

A new Gerbera plant particularly distinguished by having a novel combination of medium lengths of peduncles, semi-double bright white flower color, an overall flower diameter of approximately 11 cm, a moderate level of resistance to powdery mildew, and demonstrated potential to produce attractive plants in large (≧20-cm in diameter) containers, is disclosed.

### GENUS AND SPECIES

Gerbera hybrida

### VARIETY DENOMINATION

‘UFGE 7014’

### BACKGROUND OF THE NEW PLANT

The invention relates to a new and distinct variety of Gerbera hybrida plant named ‘UFGE 7014’. ‘UFGE 7014’ originated from a cross made at Wimauma, Fla. in 2005 between the female parent, ‘Sunburst Snow White’ (unpatented), and the male parent, ‘UFGE 4033’ (unpatented), an unreleased breeding line selected at Wimauma, Fla. in 2004 from a population of progeny of the cross ‘UFGE 31-19’ (unpatented) and ‘UFGE 35-4’ (unpatented). ‘UFGE 7014’ was selected by the inventors from the progeny of the stated parentage in summer 2007 at Wimauma, Fla. The first asexual reproduction of ‘UFGE 7014’ was accomplished when crown division was done in fall 2007 at Wimauma, Fla. Plants of ‘UFGE 7014’ have been asexually propagated by crown division and/or tissue culture for more than three generations. Asexually propagated plants of ‘UFGE 7014’ have remained true to the original selected plant, and all characteristics of the plant have been transmitted and retained through three successive asexual vegetative generations.

Plant Breeder's Rights for this cultivar have not been applied for. ‘UFGE 7014’ has not been made publicly available more than one year prior to the filing of this application.

### SUMMARY OF THE INVENTION

The new and distinct variety of Gerbera plant is characterized by a novel combination of medium lengths of peduncles, semi-double bright white flowers, an overall flower diameter of approximately 11 cm, a moderate level of resistance to powdery mildew, and demonstrated potential to produce attractive plants in large (≧20-cm in diameter) containers. ‘UFGE 7014’ has not been observed under all possible environmental conditions. Its phenotype may vary significantly with variations in environment such as light intensity, temperature, and day length. The following are the most outstanding and distinguishing characteristics of this new cultivar when grown at Wimauma, Fla. under normal horticultural practices in greenhouse conditions which closely approximate those generally used in commercial practice.

1. Peduncle length of approximately 40 cm;

2. Semi-double flower type;

3. White (RHS 155D) ray florets;

4. Yellow-green (RHS 151B) color discs before opening of disc florets;

5. White (RHS 155B) perianth lobe of disc florets;

6. Flower head diameter of approximately 11 cm; and

7. Moderate resistance to powdery mildew.

### DESCRIPTION OF THE PHOTOGRAPHS

This new Gerbera plant is illustrated by the accompanying photographs which show the plant's form, flower head, and foliage. The colors shown are as true as can be reasonably obtained by conventional photographic procedures. The photographs are of a plant approximately 3 months old which was produced from one tissue culture liner and is potted in a 2.7-L container. Colors in the photograph may differ slightly from the color values cited in the detailed botanical description, which accurately describe the colors of the new Gerbera.

FIG. 1 shows the overall plant habit including flowers and foliage and is taken from a side perspective view.

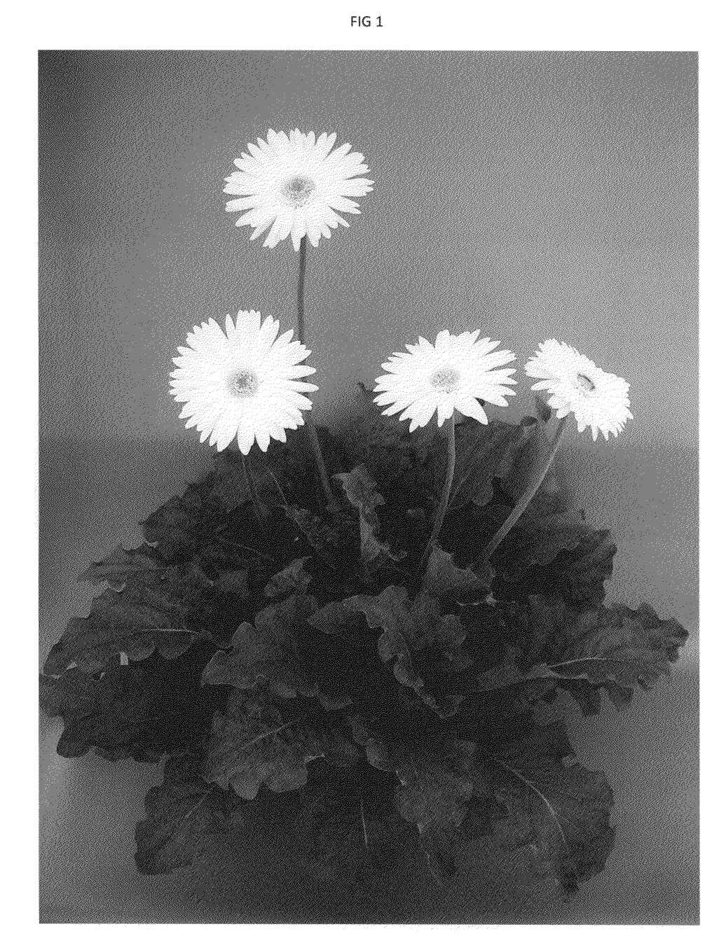

FIG. 2 shows a close-up of the flower head.

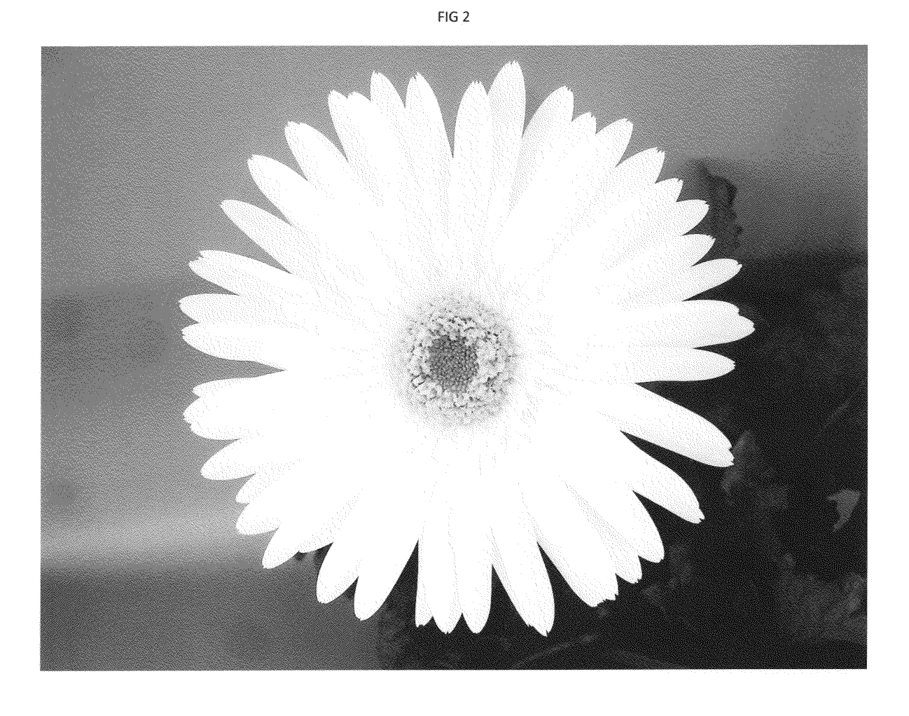

FIG. 3 shows a close-up of the typical leaf.

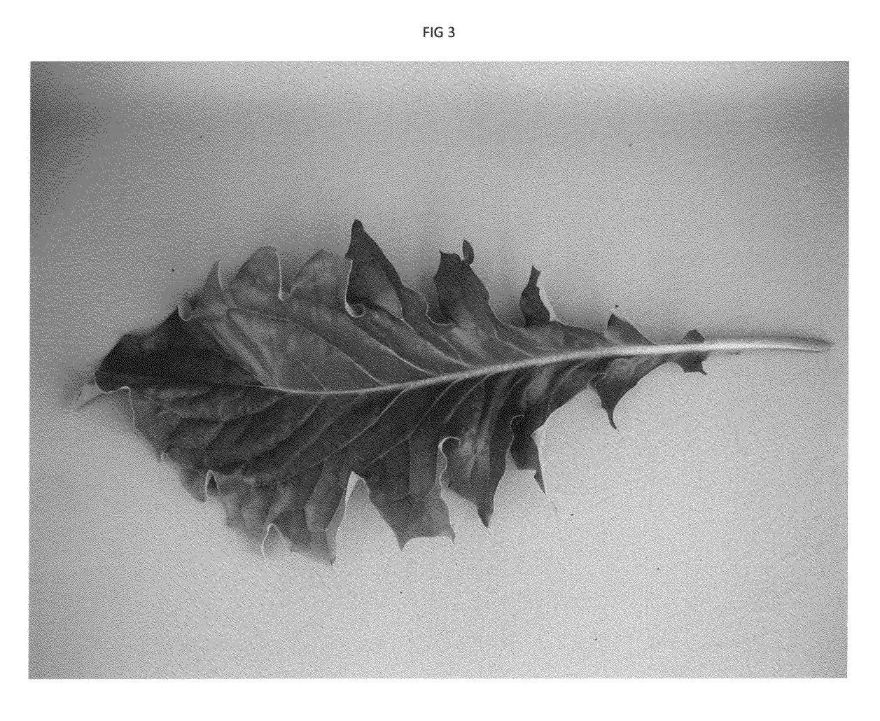

### DESCRIPTION OF THE NEW CULTIVAR

The following detailed description sets forth the distinctive characteristics of ‘UFGE 7014’. The present botanical description is that of the variety at approximately 3 months old on Oct. 20, 2009 at 11:30 a.m. in a room under north light at Wimauma, Fla. The colors (except those in common terms) are described from R.H.S. Colour Chart published by The Royal Horticultural Society in London (1986 ed.), in association with the Flower Council of Holland.

### DETAILED BOTANICAL DESCRIPTION

Classification:
   Botanical.—Gerbera hybrida cv. ‘UFGE 7014’.
   Common name.—Gerbera.
Parentage:
   Female parent.—‘Sunburst Snow White’ (unpatented).
   Male parent.—‘UFGE 4033’ (unpatented).
Inflorescence:
   Flower head.—Type: Semi-double Diameter: Large, approx. 110 mm Color (general tonality from a distance of 3 meters): Bright white (RHS 155D) Shape: Incurving funnel-shaped.
   Involucre.—Height from point of attachment of involucre to top of flower head: Short, approx. 18 mm Diameter: Small to medium, approx. 37 mm Number of bracts: Medium, approx. 101 Color: Green (RHS 138A) Longitudinal axis of inner rows: Straight Anthocyanin: Absent Pubescence: Very dense.
   Inner ray florets.—Number: Medium, approx. 61 Shape: Narrow elliptic Longitudinal axis of outer row: Moderately reflexing Longitudinal axis of inner rows: Straight Longitudinal axis of ray female floret: Moderately reflexing.
   Outer ray floret.—Cross section: Concave Length: Medium, approx. 45 mm Width: Medium, approx. 8 mm Shape of apex: Pointed Depth of incision: Medium Color (topside): White (RHS 155D) Color (bottom side): White (RHS 155B) Color distribution on inner side: Uniform Edge of different color: Absent Striation: Absent.
   Disc florets.—Disc diameter: Medium, approx. 24 mm.
   Reproductive organs.—Style (main color of distal part): White (RHS 155D) Stigma (main color): White (RHS 155D) Anthers (main color): Yellow (RHS 12A) Color of top relative to other parts: Lighter Longitudinal stripes: Absent.
   Pappus.—Main color: Yellow-white (RHS 158D) Color of top relative to other parts: Same Level of top relative to closed disc florets: Same.
   Peduncle.—Length: Medium, approx. 42 cm Tendency to fasciation: Absent Thickness: Medium, approx. 7 mm Strength: Medium Pubescence: Medium Color: Yellow-green (RHS 144B) Anthocyanin coloration: At base: Very weak, (RHS 152C) At top: Absent Involucral bracts: Absent.
Plant:
   General appearance.—Height: Approx. 42 cm, excluding any flowers Width: Approx. 57 cm.
   Foliage.—Leaf blade: Length: Long, approx. 26 cm Width: Medium, approx. 14 cm Thickness: Medium Blistering: Medium Pubescence (On upper side, midrib excluded): Sparse Depth of incisions in leaf: Basal part: Deep Central part: Medium Distal part: Shallow Color: Upper side: Yellow-green (RHS 146A) Bottom side: Yellow-green (RHS 147C) Glossiness on upper side: Strong to medium Shape of apex: Right angle Petiole: Petiole length: Medium, approx. 7.5 cm Color of petiole: Yellow-green (RHS 144B) Petiole anthocyanin coloration: Strong.
Resistance to diseases: ‘UFGE 7014’ has been observed in multiple experiments in which it was compared with two common commercial cultivars, ‘Pensacola’ gerbera (unpatented) and ‘Bimini’ gerbera (unpatented), for the incidence and severity of powdery mildew, Podosphaera (syn. Sphaerotheca) fusca (Fr.) S. Blumer. ‘UFGE 7014’ has consistently been found to have a moderate level of resistance to powdery mildew. In two experiments conducted, Experiment 1 in winter 2008 and spring 2009, and Experiment 2 in summer and fall 2009, both at Wimauma, Fla., tissue culture liners of ‘UFGE 7014’, ‘Bimini’, and ‘Pensacola’ were transplanted on into 2.7-L containers filled with commercial potting mix amended with controlled release fertilizer at the rate of 5.28 kg·m⁻³ and trace element fertilizer at the rate of 1.05 kg·m⁻³. Plants were grown under a plastic tunnel within a screen house with approximately 45% light exclusion. Fungicides were not applied throughout the experiments to subject the plants to natural powdery mildew disease pressures. In Experiment 1, the severity of powdery mildew on gerbera leaves was assessed at week 8 after transplanting (Feb. 4, 2009), week 10 after transplanting (Feb. 18, 2009), week 12 after transplanting (Mar. 4, 2009), and week 14 after transplanting (Mar. 18, 2009). In Experiment 2, the severity of powdery mildew was assessed at week 8 after transplanting (Oct. 15, 2009), week 10 after transplanting (Oct. 29, 2009), and week 12 after transplanting (Nov. 12, 2009). A randomized complete block design was used with five replications in Experiment 1 and with eight replications in Experiment 2. The experimental unit was a single containerized plant. ‘UFGE 7014’ showed a moderate level of resistance to powdery mildew in both Experiment 1 and Experiment 2 (Table 1). ‘UFGE 7014’ remained to be more productive than ‘Bimini’ and ‘Pensacola’ when natural powdery mildew disease pressure existed (Table 1). No other disease resistance characterizations have been made.

TABLE 1
Powdery mildew severity ratings and flower counts of ‘UFGE 7014’ and
two commercial gerbera cultivars, ‘Bimini’ and ‘Pensacola’, grown
under natural powdery mildew pressure in two experimental trials
in Wimauma, FL. Experiment 1 (Winter 2008 through Spring
2009), Experiment 2 (Summer 2009 through Fall 2009)

| | PM Ratings^{Z} | | | | Flower |
|---|---|---|---|---|---|
| Cultivars | Week 8 | Week 10 | Week 12 | Week 14 | counts^{Y} |
| | Experiment 1 (Winter 2008 through Spring 2009) | | | | |
| UFGE 7014 | 1.2 | 2.2 | 4.2 | 6.6 | 5.6 |
| Bimini | 1.2 | 1.8 | 5.4 | 8.0 | 3.2 |
| Pensacola | 1.0 | 2.0 | 4.8 | 8.6 | 3.0 |
| | Experiment 2 (Summer 2009 through Fall 2009) | | | | |
| UFGE 7014 | 1.5 | 6.5 | 7.0 | | 4.3 |
| Bimini | 2.6 | 8.6 | 9.4 | | 2.6 |
| Pensacola | 1.8 | 7.5 | 9.8 | | 1.1 |

^{Z}Powdery mildew severity was rated on a scale of 1 to 10 as described by Hausbeck et al. (2002): 1 = no disease, 2 = trace to 10%, 3 = 10% to 20%, 4 = 20 to 30%, 5 = 30% to 40%, 6 = 40% to 50%, 7 = 50% to 60%, 8 = 60% to 70%, 9 = 70% to 80%, and 10 = 80% to 100% of leaf surface covered with powdery mildew.
^{Y}Total number of flowers produced per plant over nine weeks from Jan. 20, 2009 to Mar. 17, 2009 in Experiment 1 or from Sep. 25, 2009 to Nov. 20, 2009 in Experiment 2, both in a screen house in Wimauma, FL

### COMPARISON WITH PARENTAL AND KNOWN CULTIVARS

‘UFGE 7014’ differs from the female parent ‘Sunburst Snow White’ (unpatented) by having larger leaves, longer peduncles, larger flowers, and stronger powdery mildew resistance under growing conditions in Wimauma, Fla. than ‘Sunburst Snow White’. ‘UFGE 7014’ differs from the male parent ‘UFGE 4033’ (unpatented) by having more leaves, fuller plants, and more flowers under growing conditions in Wimauma, Fla. than ‘UFGE 4033’.

Comparisons were made with the commercial gerbera variety ‘Bimini’ (unpatented) and the commercial gerbera variety ‘Pensacola’ (unpatented). Two experiments were conducted in a greenhouse at Wimauma, Fla. under standard greenhouse management practices to evaluate the plant performance (days to flower, flower quality, flower count, and plant quality) of ‘UFGE 7014’ for container plant production in comparison to ‘Bimini’ and ‘Pensacola’. In the first of the two plant performance experiments (Experiment 3), tissue culture liners were transplanted on Dec. 9, 2008 into 2.7-L containers filled with commercial potting mix amended with controlled release fertilizer at the rate of 5.28 kg·m⁻³ and trace element fertilizer at the rate of 1.05 kg·m⁻³. Potted plants were grown on metal benches in a glass house and spaced 46 cm×46 cm apart. Temperatures inside the greenhouse ranged from 18° C. to 33° C. Two hours of additional photoperiodic lighting were provided between Dec. 9, 2008 and Mar. 17, 2009. Fungicides and insecticides were applied as needed to control powdery mildew and western flower thrips. Flower quality was rated on a 1 to 5 scale: 1=very poor, 3=fair, some blemishes, but acceptable, and 5=excellent, bright, uniform, and no blemishes. Flower counts were recorded weekly from Jan. 20, 2009 through Mar. 17, 2009. Plant quality was rated three times, on Jan. 20, 2009, Feb. 3, 2009, and Feb. 17, 2009, using a 1 to 5 scale: 1=few leaves or long petioles, container surface visible, very poor and unacceptable as flowering pot plants, 3=fair and marketable, and 5=excellent, full, symmetrical, attractive plants.

In the second of the two plant performance experiments (Experiment 4), tissue culture liners were transplanted on Aug. 13, 2009 into 2.7-L plastic containers filled with commercial potting mix amended with controlled release fertilizer at the rate of 5.28 kg·m⁻³ and trace element fertilizer at the rate of 1.05 kg·m⁻³. Plants were grown on metal benches in a greenhouse with a spacing of 46 cm×46 cm. The greenhouse was covered with aluminum shading cloth with approximately 30% light exclusion. Greenhouse temperatures ranged from 21° C. to 35° C. Two hours of additional photoperiodic lighting was provided beginning Sep. 15, 2009. Fungicides and insecticides were applied as needed to control powdery mildew and western flower thrips. Flower quality and plant quality were rated as above for Experiment 3, but on Sep. 25, 2009, Oct. 15, 2009, and Nov. 5, 2009. The number of flowers produced per plant was recorded weekly from Sep. 25, 2009 through Nov. 20, 2009.

The experiment design for the plant performance experiments was a randomized complete block design with five replications in Experiment 3 and eight replications in Experiment 4. The experimental unit was a single containerized plant.

‘UFGE 7014’ came into flowering 14 to 19 days earlier than ‘Bimini’ and ‘Pensacola’ in Experiment 3 and 7 to 13 days earlier than ‘Bimini’ and ‘Pensacola’ in Experiment 4 (Table 2). Flower quality ratings of ‘UFGE 7014’ were 5.0 to 4.7, similar to that of ‘Bimini’ (5.0 to 4.8), and higher than that of ‘Pensacola’ (4.1 to 4.3) (Table 2). In both Experiment 3 and Experiment 4, ‘UFGE 7014’ produced more flowers (253% to 163% more than ‘Bimini’ and 543% to 360% more than ‘Pensacola’) (Table 2). The plant quality rating of ‘UFGE 7014’ was 4.7 in both Experiment 3 and Experiment 4, higher than that of the controls (Table 2).

TABLE 2
Plant performance of ‘UFGE 7014’ and two commercial cultivars,
‘Bimini’ and ‘Pensacola’ grown in Experiment 3 (Winter 2008
through Spring 2009) and Experiment 4 (Summer 2009
through Fall 2009) in Wimauma, Florida.

| | Days to | | Flower | |
|---|---|---|---|---|
| | 1st open | Flower | counts^{Z} | Plant |
| Cultivars | flowers (no.) | quality^{Z} | (no.) | quality^{Y} |
| | Experiment 3 (Dec. 9, 2008 to Mar. 17, 2009) | | | |
| UFGE 7014 | 49.6 | 5.0 | 7.6 | 4.7 |
| Bimini | 64.0 | 5.0 | 3.0 | 3.1 |
| Pensacola | 68.8 | 4.1 | 1.4 | 3.3 |
| | Experiment 4 (Aug. 13, 2009 to Nov. 20, 2009) | | | |
| UFGE 7014 | 42.3 | 4.7 | 9.0 | 4.7 |
| Bimini | 50.1 | 4.8 | 5.5 | 3.2 |
| Pensacola | 55.8 | 4.3 | 2.5 | 3.3 |

^{Z}Flower count data were collected over nine weeks from Jan. 20, 2009 to Mar. 17, 2009 in Experiment 3 and another nine weeks from Sep. 25, 2009 to Nov. 20, 2009 in Experiment 4. Each value represents the mean of five (Experiment 3) or eight (Experiment 4) replicates.
^{Y}The plant quality rating value in Experiment 3 was the mean of five replicates over three times (Jan. 20, Feb. 3, and Feb. 17, 2009), and the plant quality rating value in Experiment 4 was the mean of eight replicates over three times (Sep. 25, Oct. 15, and Nov. 5, 2009).

### Literature Cited

Hausbeck, M. K., W. R. Quackenbush, and S. D. Linderman. 2002. Evaluation of cultivars of African daisy for resistance to powdery mildew, 2002. B&C Tests 18:O0004.

I claim:

1. A new and distinct cultivar of Gerbera plant named ‘UFGE 7014’, as illustrated and described herein.
